(19) United States
(12) Patent Application Publication
(10) Pub. No.: US 2022/0251605 A1
(43) Pub. Date: Aug. 11, 2022

(54) MEMBRANE ATTACK COMPLEXES AND USES THEREOF

(71) Applicant: The Brigham and Women's Hospital, Inc., Boston, MA (US)

(72) Inventors: Jose A. HALPERIN, Boston, MA (US); Pamela GHOSH, Boston, MA (US)

(21) Appl. No.: 17/617,731

(22) PCT Filed: Jun. 15, 2020
(86) PCT No.: PCT/US2020/037738

§ 371 Date: Dec. 9, 2021

Related U.S. Application Data
(60) Provisional application No. 62/861,420, filed on Jun. 14, 2019.

(51) Int. Cl. C12N 15/87; C07K 14/47; A61K 9/00; A61K 31/7105; A61K 31/721; A61K 45/06; A61P 35/00; A61P 27/02

(52) U.S. Cl. CPC C12N 15/87; C07K 14/472; A61K 9/0048; A61P 27/02; A61K 31/721; A61K 45/06; A61P 35/00; A61K 31/7105

(57) ABSTRACT

The invention features methods for introducing a therapeutic cargo into a cell of a subject. Such methods include the steps of (a) contacting the cell with a purified human CSb-6 and the therapeutic cargo; and (b) contacting the cell with a purified C7, C8, and C9, thereby forming a membrane attack complex (MAC) in the membrane of the cell, facilitating entry of the therapeutic cargo into the cell. Still another method involves treating neovascularization in an eye of a subject, the method comprising the steps of (a) contacting choroidal blood vessels of the eye with a purified human CSb-6 and the therapeutic cargo; and (b) contacting the choroidal blood vessels with a purified C7, C8, and C9, thereby forming MACS in cells of the choroidal blood vessels, facilitating entry of the therapeutic cargo into the cells of the choroidal blood vessels for treating neovascularization.

### CROSS-REFERENCE TO RELATED APPLICATIONS

This application claims benefit of U.S. Provisional Application No. 62/861,420, filed Jun. 14, 2019, the contents of which is incorporated herein by reference in its entirety.

### BACKGROUND OF THE INVENTION

This invention relates to delivering therapeutic macromolecules into cells in vivo.

The discovery that small interfering (siRNAs) and micro-RNAs (miRNAs (Fire et al., Nature. 1998; 391(6669):806-811)) can suppress selected genes in vivo (Ahmadzada et al., Biophys Rev. 2018; 10(1):69-86; Carthew et al., Cell. 2009:136(4):642-655) and the more recent discovery of Crisper/CAS9 gene editing technology opened the possibility of developing target-specific therapies for a variety of diseases that were previously unthinkable (Van de Veire et al., Cell. 2010; 141(1):178-190; Zhao et al., Molecular bioSystems. 2013; 9(12):3187-3198; Davidson et al., Nat Rev Genet. 2011; 12(5):329-340). However, to bring these technologies into clinical practice for treatment of human diseases, at least three key steps must be accomplished: (1) identification of target genes directly involved in the pathogenesis of the disease, (2) synthesis of siRNAs or sgRNAs specific for the target gene(s), and (3) delivery of the synthetic oligonucleotides into the target cell.

While several siRNAs are currently in clinical trials, their delivery into the target cells at effective concentrations remains a major barrier in clinical practice (Tatiparti et al., Nanomaterials (Basel). 2017; 7(4)). The siRNAs or sgRNAs must be shielded from degradation by nucleases, immune surveillance and excretion, and enabled to cross the cell membrane, an extremely inefficient process because RNAs are highly charged (Essex et al., Gene Ther. 2015; 22(3):257-266). Several clinical trials targeting angiogenesis, metastasis or the proliferation of cancer cells by systemic intravenous injection of 5 to 15 kD encapsulated oligonucleotides have failed mostly due to poor delivery into target cells (Pai et al., Gene Ther. 2006; 13(6):464-477; Miller et al., Expert review of medical devices. 2013; 10(6):781-811). Local delivery of oligonucleotides by intraocular, intra-tumoral injection or intranasal delivery are also being investigated. At this time, the only successful delivery strategy that led to an U.S. Food and Drug Administration approved drug conjugated the therapeutic siRNA to the sugar molecule GalNAc, which binds to the surface of hepatocytes. This mode of delivery requires conjugation of siRNAs to a molecule that will specifically bind to the target cell, a feature that may or may not be available for all target cells and will require developing new chemistries for every target cell type (Lawrence et al., The Pharmaceutical Journal. 2018; 300(7913):2-12).

There is accordingly a need in the art for methods and compositions allowing for the uptake and intracellular delivering of therapeutic macromolecules such as siRNAs, miRNAs, and gene-editing components into cells in vivo.

### SUMMARY OF THE INVENTION

In one aspect, the invention features a kit including (a) a purified human C5b-6 and (b) a therapeutic cargo. In preferred embodiments, (a) and (b) are provided in combination. In other embodiments, the kit further includes (c) a purified C7, (d) a purified C8 or (e) a purified C9 or any combination of (c), (d), or (e). In other embodiments, the cargo is a nucleic acid molecule (e.g., an RNA or an siRNA). In still other embodiments, the nucleic acid molecule is a component of a gene editing system. In still other embodiments, the cargo is a polypeptide or a therapeutic agent (such as a chemotherapeutic or a pharmaceutical). In yet other embodiments, the cargo is a cell impermeable molecule.

In yet another aspect, the invention features a method for introducing a therapeutic cargo into a cell of a subject, the method including the steps of (a) contacting the cell with a purified human C5b-6 and the therapeutic cargo; and (b) contacting the cell with a purified C7, C8, and C9, thereby forming a membrane attack complex (MAC) in the membrane of the cell, facilitating entry of the therapeutic cargo into the cell. In preferred embodiments, the C5b-6 and the therapeutic cargo are provided in combination. In other embodiments, the cargo is a nucleic acid molecule (e.g., an RNA or an siRNA). In still other embodiments, the nucleic acid molecule is a component of a gene editing system. In still other embodiments, the cargo is a polypeptide or a therapeutic agent (such as a chemotherapeutic or a pharmaceutical). In yet other embodiments, the cargo is a cell impermeable molecule. In yet other embodiments, the cell is a cancer cell (e.g., a pancreatic or breast cancer cell). In still other embodiments, the cell is a choroidal blood vessel cell. In preferred embodiments, the subject is a human. And in yet other preferred embodiments, MAC is autologous to the subject.

In still another aspect, the invention features a method for treating neovascularization in an eye of a subject, the method including the steps of (a) contacting choroidal blood vessels of the eye with a purified human C5b-6 and the therapeutic cargo; and (b) contacting the choroidal blood vessels with a purified C7, C8, and C9, thereby forming MACs in cells of the choroidal blood vessels, facilitating entry of the therapeutic cargo into the cells of the choroidal blood vessels for treating neovascularization. In preferred embodiments, the C5b-6 and the therapeutic cargo are provided in combination. In other embodiments, the cargo is a nucleic acid molecule (e.g., an RNA or an siRNA). In still other embodiments, the nucleic acid molecule is a component of a gene editing system. In still other embodiments, the cargo is a polypeptide or a therapeutic agent (such as a chemotherapeutic or a pharmaceutical). In yet other embodiments, the cargo is a cell impermeable molecule. In preferred embodiments, the neovascularization causes wet age-related macular degeneration. In still other preferred embodiments, contacting involves injecting the purified C5b-6, the therapeutic cargo, and the purified C7, C8, and C9 into the vitreous of the eye. In still other preferred embodiments, the subject is a human and the MACs are autologous to the subject.

The invention provides several useful therapeutic advantages. For example, the invention provides for using human MAC to deliver in vivo therapeutic macromolecules that cannot cross the cell membrane. Such therapeutic macromolecules are not limited to biological agents but also include large bio-active natural products and synthetic chemicals. Indeed, the MAC pore may be utilized to deliver components of gene editing systems such as the Crisper/CAS9 gene editing system into somatic cells to correct for inherited mutations that affect a subset of somatic cells or even mutations in germ cells. Furthermore, the MAC may be readily formed with complement components purified from each individual's own blood, allowing for the delivery of allogeneic MAC components to an individual. The described MAC systems accordingly allow for safe delivery of therapeutic agents that do not readily cross the cell membrane in native form and therefore require very high and often times toxic doses to be partially effective. The invention therefore advantageously allows for administering therapeutics at lower, less toxic formulations, reducing an individual's exposure to potentially toxic agents.

Other features and advantages of the invention will be apparent from the following description of the preferred embodiments thereof, and from the claims.

### BRIEF DESCRIPTION OF THE DRAWINGS

FIG. 1 shows that a membrane attack complex (MAC) allows uptake of macromolecules into cultured cells. HUVECs cells were exposed to fluorescent Texas-red dextran (mw:15 kD) (A) or FITC-oligonucleotides (19 bp) (B) in the absence or presence of purified terminal complement components (C5b-6, C7, C8 and C9) that form the MAC. In the presence of the MAC, all cells took up the dextran or the oligonucleotides without compromising their viability.

FIG. 2 shows that MAC-induced uptake of FITC-labeled 19 bp oligonucleotides into HUVECs in culture.

FIG. 3 is a graph showing that MAC-induces the efficient uptake of FL siRNA into 3T3 cells.

FIG. 4 shows that mouse peritoneal cells take up 20 k fluorescent dextran only in the presence of the MAC.

FIG. 5 shows that siRNA target-specific down-regulation of Gpr 77v1 in the presence but not the absence of the MAC in mouse peritoneal cells. Gpr 160V1 was used as a negative control.

### DETAILED DESCRIPTION OF THE INVENTION

Below a membrane attack complex of complement (MAC) for delivery inside target cells of therapeutic macromolecules that must but cannot cross the cell membrane is described.

The complement system is an effector of both adaptive and innate immunity. It is composed of more than 30 plasma proteins that normally circulate as inactive precursors. Complement proteins interact with one another in three enzymatic activation cascades (classical, alternative and lectin pathways) that eventually converge at the level of C3 and C5; thereafter, the three pathways share a common reaction sequence through the late components C6, C7, C8, and C9; polymerization of C9 eventually forms the MAC, the main effector of the terminal complement-pathway (see, for example, (Ghosh et al., Endocr Rev. 2015; 36(3):272-288)). The MAC is a trans-membrane pore with an effective radius of ≈7 nm and the capacity to perforate and kill “non-self cells” such as foreign erythrocytes or bacteria.

As is discussed below, we describe in vivo evidence in mice using MAC for delivering siRNAs as a representative macromolecular drug for treatment of human diseases. Uses of human MAC to deliver in vivo therapeutic oligonucleotides and/or other macromolecules are also described for (1) inhibiting human breast and pancreatic cancer by delivering siRNAs targeting cyclin D1 through MAC pores and (2) inhibiting pathological blood vessels growth in a mouse model of macular degeneration by delivering siRNAs targeting vascular endothelial growth factor (VEGF) and angiopoietin-2 (Ang-2) through the MAC pore.

MAC Allows Uptake of Macromolecules into Cultured Cells In Vitro

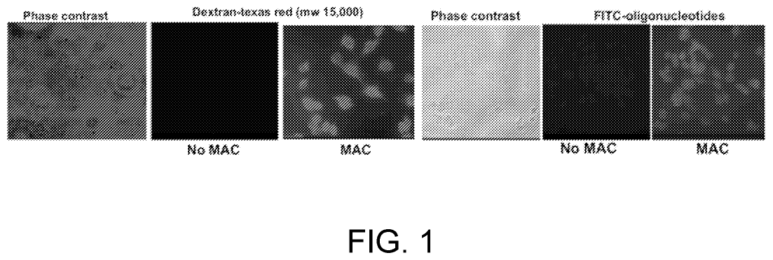

As shown in FIG. 1, formation of the transient MAC pore allows the uptake of macromolecules such a fluorescent dextrans of up to 20 kD (FIG. 1A) or FITC-labeled oligonucloteoides (FIG. 1B) in vitro (Acosta et al., Mol Med. 1996; 2(6):755-765).

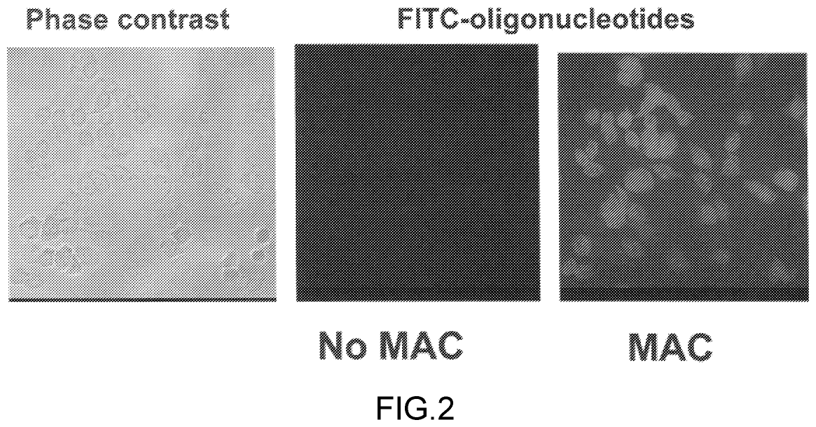

FIG. 2 shows that MAC-induced uptake of a FITC-labeled 19 bp oligonucleotides into human umbilical vein endothelial cells (HUVECs) in culture.

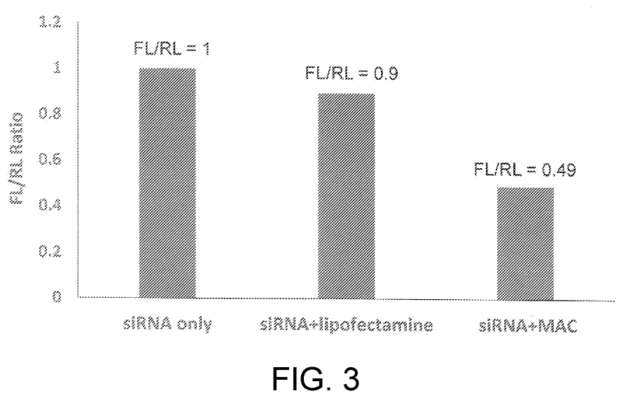

FIG. 3 is a graph showing that MAC-induces the efficient uptake of firefly luciferase (FL) siRNA into 3T3 cells. Here MAC is used as a transfection tool for siRNA (in vitro). 3T3 cells were stably transfected with a dual vector coding for both firefly (FL) and Renilla luciferase (RL) (dual luciferase assay). siRNA was used against firefly luciferase. The effect of the siRNA is determined by the ratio of FL/RL in the dual luciferase assay system. Comparison with lipofectamine was used to assess the efficiency of the MAC as a transfection tool.

MAC Allows Uptake of Macromolecules into Cultured Cells In Vivo

Transient MAC pores were found to facilitate the cellular uptake of macromolecules in vivo. We injected into the peritoneal cavity of mice a suspension containing fluorescent dextran (20 kD) and purified human C5b-6; one minute later we added purified C7, C8 and C9 which sequentially react with C5b-6 to form MAC pores on cell membranes or just PBS as a vehicle control.

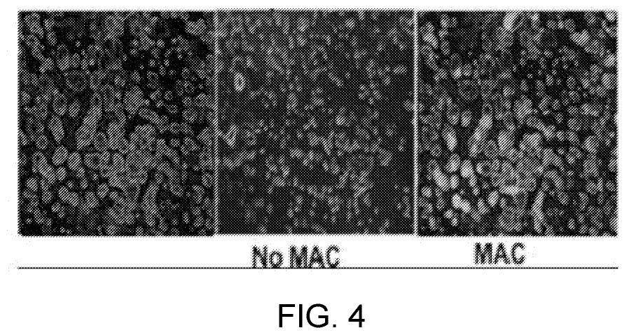

FIG. 4 shows that allowing MAC formation facilitated the uptake of 20 kD fluorescent dextran into peritoneal cells that were extracted and analyzed ex-vivo 20 minutes after the dextran injection. No uptake was found in the peritoneal cells exposed to the fluorescent dextran in the absence of the MAC.

To assess whether the MAC would also facilitate the uptake of a siRNA into peritoneal cells at effective concentrations to suppress gene expression, we repeated the study depicted in FIG. 4. but replaced dextran with a siRNA designed to target Gpr77v1, a gene expressed in mouse peritoneal cells. We assessed the expression of Gpr 77V1 (siRNA target) or 106v1 (negative control) by RT-PCR.

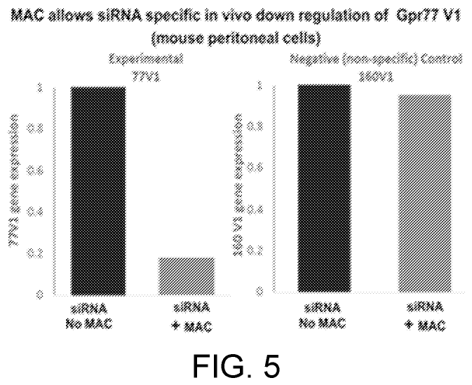

FIG. 5 shows that peritoneal cells exposed in vivo to the 77v1 targeting siRNA in the presence of the MAC specifically knocked-down its expression while the same amount of siRNA injected without the MAC was ineffective. Another gene of the same family commonly expressed in mouse peritoneal macrophages (160v1) was not affected by the siRNA targeting 77v1, confirming that the gene knock down was specific for the targeted gene rather than a non-specific consequence of MAC formation. In this example, siRNA designed against Mice Gpr77 TV1 (C5ar2 TV1) (G-protein receptor 77, transcript variant 1). The negative control for non-specific effect was analysis of expression of Gpr160 TV1 (G protein receptor 160, transcript variant 1). Both genes were expressed in mouse peritoneal macrophages. siRNA was injected by intraperitoneal injection (ip) with or without terminal C components to form the MAC. Expression of either Gpr77 TV1 (experimental) or Gpr160 TV1 (negative control for non-specific effect) in peritoneal cells was quantified 24 hours later by RT-PCR according to standard methods.

### Inhibition of Human Breast and Pancreatic Cancer Using MAC Pores

siRNA targeting cyclin D1 is used because it drives cell cycle progression through the G1 phase by forming dimers with cyclin dependent kinases (Cdks) 4 and 6. Cyclin D1 expression is induced by mitogens that signal through the Ras proto-oncogene and is required for Ras-dependent cellular proliferation and transformation (Aktas et al., Mol Cell Biol. 1997; 17(7):3850-3857). In addition to its catalytic functions in the cyclin-cdk4/6 complexes, Cyclin D1 interacts with over 100 other proteins contributing to transcription, DNA damage repair, and cell migration (Musgrove et al., Nat Rev Cancer. 2011; 11(8):558-572).

Cyclin D1 is also one of the most frequently altered proto-oncogenes in human cancers, and increased cyclin D1 expression is associated with poor prognosis. In many human cancers including breast, pancreas, colon, and lung, the cyclin D1 gene is amplified, fused to promoters that increase its expression or mutated/truncated in a manner that increases cyclin D1 accumulation. Also, mutations of several other proto-oncogenes increase the stability or nuclear export of cyclin D1 protein. Chemical inhibitors of cyclin-cdk4/cdk6 kinases have shown a significant potential in preclinical models of cancer and in clinical trials; however, off target effects as well as acquired resistance are drawbacks that reduce the clinical utility of these agents.

Targeted suppression of cyclin D1 expression by administration of cyclin D1 specific siRNAs delivered into cells through transient MAC pores circumvents these drawbacks because the siRNAs will abrogate both catalytic and non-catalytic functions of cyclin D1 reducing the risk of target-independent drug resistance.

### In Vivo Model of Breast Cancer

Most breast cancers, including those driven by EGFR, HER2/neu or estrogen receptor signaling are dependent on cyclin D1 for survival and progression. In vivo efficacy of MAC-mediated delivery of cyclin D1 specific siRNA to inhibit growth of human breast cancer tumors using a fluorescence tagged MCF-7 breast cancer cell line is accordingly then evaluated. This cell line, which is estrogen receptor positive/HER2 expression normal, which is tagged with green fluorescent protein (GFP) will be utilized. In these studies, tumors in nude female mice will be formed by ip.

The ip route is chosen for these studies because it facilitates the injection and uniform distribution of reagents, namely MAC forming proteins and siRNA. Because MCF-7 tumors are estrogen dependent, three days before tumor cell injection into the peritoneal cavity, female nude mice are implanted with a subcutaneous slow release estrogen pellet; then 107 GFP tagged MCF-7 cells in 50% matrigel will be injected into the peritoneal cavity. Five days later, siRNAs targeting cyclin D1 or mock siRNA and purified human C5b-6 are injected followed one minute later by injection of purified C7, C8 and C9 to form the MAC or just PBS as a vehicle control. Table 1 summarizes our experimental design in 4 animal groups.

TABLE 1

| | |
|---|---|
| Cyclin D1 siRNA + C5b-6 | Mock siRNA + C5b-6 |

| | | | |
|---|---|---|---|
| C7, C8, C9 | PBS | C7, C8, C9 | PBS |

To evaluate uptake of siRNAs into the cells, tracer amounts of Texas-red labeled cyclin D1 specific or mock siRNA are mixed. Every day after, 200 μl of PBS are injected ip and then aspirated peritoneal fluid containing cancer cells is collected. For uptake of siRNAs cells analyzed under the fluorescent microscope similar to the experiment depicted in FIG. 2. The knock-down efficiency of cyclin D1 specific or mock siRNAs by RT-PCR and staining for cyclin D1 protein and the proliferation marker Ki-67 is also evaluated.

In one example, the uptake of siRNA using the human terminal complement components in the concentrations already established in the experiment depicted in FIG. 5 is determined. The uptake of Texas red labeled tracer siRNAs permits determination of whether the concentration of reagents is appropriate or is to be optimized. The knock down efficiency is determined daily in the aspirated cells by RT-PCR comparing the cyclin D1 specific or the mock siRNA treated cells. Injections are repeated once the cyclin D1 mRNA recovers to 50% of its pre-knockdown level. This protocol is repeated and standard pharmaco-dynamic modeling to determine the interval at which mice should be re-injected with siRNAs and complement components to maintain steady state knockdown efficiency of Cyclin D1 mRNA and protein above 70% is determined. This method for development studies is utilized with three mice per group.

After optimizing the reagents, concentrations, and injection intervals, the efficacy of MAC-mediated uptake of cyclin D1 specific siRNA in inhibiting breast cancer growth is assessed. To this end, experiments with 10 mice in each of the 4 groups shown in Table 1 is to be conducted. Tumor growth is then assessed weekly by whole body imaging using a Bruker In-Vivo Extreme II Optical/X system that provides full spectrum bioluminescence and fluorescence imaging capabilities. At the time of imaging, peritoneal cells are aspirated from two mice in each group (rotating the mice) to ascertain the efficient knock-down of cyclin D1 mRNA by RT-PCR and suppression of cell proliferation by staining for the cell proliferation marker Ki-67. The experiment is terminated if a) >15% of mice in any group die or must be sacrificed because the tumor burden exceeds 15% of body weight as determined by total fluorescent volume or b) 6 weeks after the injection of cancer cells. Twenty-four hours before termination, all animals will be injected with BrdU to label newly synthesized DNA as a marker of cell proliferation. At the conclusion of the experiment, tumors will be removed and weighted.

In Vivo Model of Pancreatic Cancer

Similar to most breast cancers, survival and progression of most pancreatic cancers is dependent on cyclin D1 expression. Consistently, cyclin D1 levels negatively correlate with patient survival. The K-Ras proto-oncogene sustains activating mutations in significant proportion of pancreatic cancers. In many of the same tumors, the catalytic subunit of phosphatidyl inositol 3-kinase (PIK3CA) is also mutated; both K-Ras and PIK3CA induce cyclin D1 expression.

In this example, pancreatic tumors are accessed through a single artery thus facilitating administration without systemic administration. Using a pancreatic cancer model the experimental approach described above for the breast cancer model example is followed except that i) both male and female mice are used, ii) estradiol pellets are not transplanted, and iii) mice with GFP tagged Panc-1 pancreatic cancer cells are injected.

Statistical Analysis

The main end-point of these studies is inhibition of tumor growth as assessed by fluorescent imaging in vivo and by tumor weight at the end of the experiment. Injection of siRNAs targeting cyclin D1 in the presence of the MAC will significantly reduce tumor growth as compared to administration of the same siRNA with PBS or injection of mock siRNA with or without MAC. The T/C % (T=mean tumor size in treated group; and C=mean tumor size in vehicle control group and T/C %=T/C*100) is calculated following NCI protocols. Comparisons of mean±standard deviation of T/C % is analyzed by two-way ANOVA testing for cyclin D1 siRNA effect, MAC effect and interaction of the two. Alternatively, data is analyzed by multiple regression analysis using a general linear model. This analysis permits determination of the relative contribution of each variable to tumor growth. In all cases, the null hypothesis is tested; namely, that there are no differences between the groups, setting the criterion for significance at 0.05. To estimate the number of animals to yield a statistically significant result, it will be assumed that the smallest effect that would be important to detect would be a T/C of 50%, and a common within-group standard deviation not higher than 15%. Under these assumptions and with a sample size of 10 animals per group, these studies will have a power of more than 80% to yield a statistically significant result at a confidence level of 95%. Our secondary endpoint is staining for the Ki-67 proliferation marker, confirmed by BrDU and Cyclin D1 staining, which will be analyzed by Mann-Whitney (Wilcoxon) nonparametric tests.

Treatment of Age Related Macular Degeneration (AMD)

Age-related macular degeneration (AMD), a leading cause of blindness, occurs in two forms: exudative (“wet”) and nonexudative (“dry”) AMD.

In the “wet” type AMD, abnormal blood vessels grow under the retina and macula from the choroidal vasculature through breaks in the Bruch's membrane and toward the outer retina, a pathology known as choroidal neovascularization or CNV. These neovessels are immature and leaky, resulting in subretinal and intraretinal edema and hemorrhage, which leads to vision loss. The current standard of care for patients with CNV is to inhibit the pro-angiogenic and permeability molecule vascular endothelial growth factor-A (VEGF) with the anti-VEGF agents Lucentis and Eylea. Anti-VEGF therapy blocks vascular permeability and angiogenesis, but does not result in vascular regression. Patients have to receive an intravitreal injection every 1-2 months and continue indefinitely, creating a significant treatment burden to prevent further disease progression. While substantial vision improvement occurs in one-third, one-sixth of treated patients still progress to legal blindness. Combination therapy utilizing siRNA knockdown of key proteins (VEGF/Ang2) that drive this pathology would be of significant clinical interest. Use of the MAC as an effective tool to deliver intracellular siRNA represents a paradigm shift towards promising clinical applications that could bring a direct benefit to individuals with this debilitating disease.

Animal Model of AMD

To evaluate MAC-mediated delivery of siRNAs targeting VEGF/Ang2 to inhibit neovascularization, the standard well-defined mouse model of laser-induced CNV (Hasegawa et al., PNAS 2017; 114(36):E7545-E7553; Yanai et al., PNAS 2014; 111(26):9603-9608) is utilized. C57BL/6 mice (8 weeks old) will undergo CNV induction. Twenty-four hours after laser injury, mice will receive an intra-vitreal injection of C5b-6 together with siRNA targeting both VEGF-A and Ang-2 (Ryoo et al., Nanoscale. 2017; 9(40):15461-15469; D'Souza et al., J Cancer Res Clin Oncol. 2012; 138(12):2017-2026). Immediately after, C7, C8 and C9, which form the MAC, or PBS will be injected. All groups are compared to a mock siRNA.

At 3, 5, 7, and 14 days CNV induction, the lesion area using whole-mount staining of the vasculature with isolectin-488, SD-OCT is assessed, and assessment of vascular leakage with fluorescein angiography in both males and females (Hasegawa et al., PNAS 2017; 114(36):E7545-E7553; Hasegawa et al., PLoS One. 2014; 9(9):e106507), as described by Giani et al. (Invest Ophthalmol Vis Sci. 2011; 52(6):3880-3887) and Montezuma et al. (Semin Ophthalmol. 2009; 24(2):52-61) is performed. AMD disease severity with sample type masked to the assessor is to be quantified using a computer-aided quantification macro protocol in the software program, ImageJ (Maidana et al., Invest Ophthalmol Vis Sci. 2015; 56(11):6701-6708). It is expected that a 90% power to detect significant differences in the severity of disease using an unpaired t-test with an estimated standard deviation (SD) of 10 and a significance level (p value) of 0.05 is achieved.

Evaluation of Choroidal Neovascularization (CNV) Development

Choroidal/Retinal Flat Mounts

In this working example, the entire retina is carefully dissected from the eyecup. Radial cuts are made from the edge of the eyecup to the equator, and the preparation is then stained overnight with Alexa Fluor 488-conjugated to Griffonia simplicifolia isolectin B4 to visualize the vasculature. Fluorescence images of choroidal flat-mounts are captured with a Zeiss AxioObserver.Z1 microscope. CNV area is measured with ImageJ software (NIH).

Spectral Domain Optical Coherence Tomography (SD-OCT)

SD-OCT (Bioptigen Inc.) is performed as described by Giani et al., Invest Ophthalmol Vis Sci. 2011; 52(6):3880-3887). The software generates an en face fundus image with the reflectance information obtained from the OCT sections (volume intensity projection). For evaluation of the cross-sectional size of each lesion, OCT image sections passing through the center of the CNV lesion are chosen.

Fluorescein Angiography

Fluorescein angiography (FA) is performed using a Micron IV mouse-imaging system as described by (Yu et al., Invest Ophthalmol Vis Sci. 2008; 49(6):2599-2605). Fluorescein angiograms in a masked manner at a single sitting are evaluated according to standard procedures.

### OTHER EMBODIMENTS

Other embodiments are within the following claims. For example, the order of adding C5b-6, C7, C8, and C9 for forming MAC is not necessarily sequence dependent with the exception of adding C5b-6 with C7 before adding other MAC components. Other combinations are accordingly useful, for example, C7, C8 and C9 may be added first and then C5b-6, or C5b-6 and C8 and C9 may be added first and then C7.

1. A method for introducing a therapeutic cargo into a cell of a subject, the method comprising the steps of (a) contacting the cell with a purified human C5b-6 and the therapeutic cargo; and (b) contacting the cell with a purified C7, C8, and C9, thereby forming a membrane attack complex (MAC) in the membrane of the cell, facilitating entry of the therapeutic cargo into the cell.

2. The method of claim 1, wherein the C5b-6 and the therapeutic cargo are provided in combination.

3. The method of claim 1, wherein the cargo is a nucleic acid molecule.

4. The method of claim 3, wherein the nucleic acid is RNA.

5. The method of claim 4, wherein the RNA is siRNA.

6. The method of claim 3, wherein the nucleic acid molecule is a component of a gene editing system.

7. The method of claim 1, wherein the cargo is a polypeptide.

8. The method of claim 1, wherein the cargo is a therapeutic agent.

9. The method of claim 8, wherein the therapeutic agent is a chemotherapeutic.

10. The method of claim 8, wherein the therapeutic agent is a pharmaceutical.

11. The method of claim 1, wherein the cargo is a cell impermeable molecule.

12. The method of claim 1, wherein the cell is a cancer cell.

13. The method of claim 12, wherein the cancer is pancreatic or breast cancer.

14. The method of claim 1, wherein the cell is a choroidal blood vessel cell.

15. The method of claim 1, wherein the subject is a human.

16. The method of claim 1, wherein the MAC is autologous to the subject.

17. A method for treating neovascularization in an eye of a subject, the method comprising the steps of (a) contacting choroidal blood vessels of the eye with a purified human C5b-6 and the therapeutic cargo; and (b) contacting the choroidal blood vessels with a purified C7, C8, and C9, thereby forming MACs in cells of the choroidal blood vessels, facilitating entry of the therapeutic cargo into the cells of the choroidal blood vessels for treating neovascularization.

18. The method of claim 17, wherein the C5b-6 and the therapeutic cargo are provided in combination.

19. The method of claim 17, wherein the neovascularization causes wet age-related macular degeneration.

20. The method of claim 17, wherein contacting involves injecting the purified C5b-6, the therapeutic cargo, and the purified C7, C8, and C9 into the vitreous of the eye.

21. The method of claim 17, wherein the subject is a human.

22. The method of claim 17, wherein the MACs are autologous to the subject.

23.-36. (canceled)
